(19) United States
(12) Patent Application Publication
(10) Pub. No.: US 2023/0190623 A1
(43) Pub. Date: Jun. 22, 2023

(54) COSMETIC NAIL CARE COMPOSITION IN THE FORM OF A VARNISH

(71) Applicant: FIABILA, MAINTENON (FR)

(72) Inventors: Arnaud PAILOT, SPINCOURT (FR); Christine RENARD, MAINTENON (FR); Charlotte ROUF, BAILLY-ROMAINVILLIERS (FR)

(21) Appl. No.: 17/925,565

(22) PCT Filed: May 18, 2021
(86) PCT No.: PCT/FR2021/050863

§ 371 Date: Nov. 15, 2022

(30) Foreign Application Priority Data
May 19, 2020 (FR) 20 05017

(51) Int. Cl. A61K 8/73; A61K 8/60; A61Q 3/02; A61K 8/9789

(52) U.S. Cl. CPC A61K 8/73; A61K 8/60; A61K 8/9789; A61Q 3/02

(57) ABSTRACT

The invention relates to a cosmetic nail care composition in the form of a varnish, which is to be applied to the surface of the nail, characterized in that it contains an aqueous chestnut extract Castanea sativa. The invention also relates to the use of said cosmetic composition for improving the surface condition of the nail, in particular for nourishing and hydrating the nail.

The present invention relates to the field of cosmetic compositions for the nails, more particularly to the field of cosmetic compositions existing in the form of a varnish, and to the use of these cosmetic compositions for improving the surface condition of the nails.

### STATE OF THE ART

Nail varnish formulators have long sought to incorporate, into said varnishes, substances or molecules which can improve the surface condition of the nails.

Thus, in order to strengthen or harden weakened, brittle, split or cracked nails, varnish compositions comprising aldehydes or acids have been proposed.

However, the incorporation of formaldehyde, which has been known for a long time, is now ruled out because of its toxicity and its allergenic nature. The same is true for the use of glyoxal (described in the patent GB 2 196 978) or citral, described as a hardener in the patent EP 1 408 917, which is also allergenic.

As for acids, such as, for example, α-hydroxy acids, they often cause irritation of the skin surrounding the nail, and are moreover difficult to stabilize in varnish compositions.

The current trend is also to seek to use substances of natural origin, rather than synthetic molecules. However, these substances must be compatible with the organic or aqueous solvent media of nail varnishes. Moreover, they must not have an adverse impact on the qualities of these varnishes.

### AIMS OF THE INVENTION

A first aim of the invention is thus to provide a nail care composition existing in the form of a varnish containing an active principle of natural origin, which does not exhibit the abovementioned disadvantages.

Another aim of the invention is to provide a nail care composition existing in the form of a varnish which is stable over time, at the concentrations of the active principle at which the latter is effective as nail care, including for different shades of varnish. The term “nail care” is understood in particular to mean an improvement in the condition of the surface of the nail.

Another aim of the invention is to provide a nail care composition existing in the form of a varnish containing an active principle of natural origin which does not degrade the properties of gloss, of adhesion, of hardness and of stability of the base varnish (that is to say, of a varnish not containing said active principle).

### DESCRIPTION OF THE INVENTION

In the search for a substance or molecule which can overcome the above disadvantages, the inventors have discovered, surprisingly, that the incorporation, in a nail varnish composition, of rhamnogalacturonan and uronic acids, in particular resulting from a chestnut extract, could meet the abovementioned aims.

To this end, the present invention provides a cosmetic nail care composition existing in the form of a varnish to be applied to the surface of the nail, characterized in that it contains at least one rhamnogalacturonan and at least one uronic acid.

The cosmetic nail care composition can contain a chestnut extract, preferably a Castanea sativa extract, comprising rhamnogalacturonans and uronic acids.

Preferably, said composition comprises an amount of chestnut extract of between 0.01 % and 10% by weight, preferably of between 0.05% and 8% by weight, preferably of between 0.1% and 6% by weight.

The chestnut extract is preferably an aqueous extract prepared according to the process described in the French patent FR 2 854 800 and sold under the name Recoverine® by the company SILAB and containing between 8% and 12% by weight of dry matter. This extract comprises in particular rhamnogalacturonans and uronic acids. It can be used in the composition according to the invention in aqueous form or in dry form.

This care active principle has proved to be particularly advantageous, by acting on the keratinocytes of the nail, in improving the surface condition of the nail, in particular in nourishing and hydrating the nail, without causing irritation of the skin in test subjects or causing other adverse side effects.

It also turns out that this aqueous chestnut extract can be incorporated either in solvent-based varnishes, that is to say in varnishes comprising a predominantly (more than 50% by weight) solvent medium formed of organic solvents, or in aqueous varnishes, that is to say the solvent medium of which comprises predominantly water (more than 50% by weight).

According to a first embodiment of the invention, the nail care composition is characterized in that the varnish is a solvent-based varnish comprising an organic solvent medium, at least one film-forming agent, at least one plasticizer, at least one resin and optionally a rheology agent and contains said chestnut extract in a proportion of between 0.01 % and 1 % by weight, preferably of between 0.05% and 0.8% by weight, more preferably of between 0.1% and 0.6% by weight.

It appeared that, at such concentrations, the chestnut extract does not affect the stability of the varnish in comparison with a varnish without chestnut extract, in particular, as will be seen in the examples, neither the sedimentation nor the syneresis of the varnish.

The organic solvent medium can comprise one or more solvents chosen from: short-chain esters having from 3 to 8 carbon atoms in total, such as ethyl acetate, methyl acetate, propyl acetate, isopropyl acetate, butyl acetate, isopentyl acetate, methoxypropyl acetate or butyl lactate, ketones which are liquid at ambient temperature, alcohols, such as ethanol, isopropanol, butanol or diacetone alcohol, linear or branched aliphatic hydrocarbons which are saturated, such as alkanes, or unsaturated, and liquid at ambient temperature, aromatic hydrocarbons, such as toluene or xylene, and their mixtures.

Preferably, the organic solvent medium comprises ethyl acetate and/or butyl acetate and/or at least one alcohol chosen from ethanol, isopropanol and butanol.

According to a second embodiment of the invention, the composition is characterized in that the varnish is an aqueous-based varnish comprising an aqueous solvent medium, at least one film-forming agent, at least one plasticizer, at least one resin and optionally a rheology agent and contains said chestnut extract in a proportion of between 0.01 % and 10% by weight, preferably of between 0.05% and 8% by weight, more preferably of between 0.1% and 6% by weight.

According to the present invention, the varnish comprises at least one film-forming agent which is preferably a cellulose film-forming agent, chosen from nitrocellulose, carboxymethylcellulose, hydroxyethylcellulose, hydroxypropylcellulose, cellulose acetate butyrate and/or cellulose acetate propionate.

The varnish can also comprise one or more monomers or oligomers based on (meth)acrylate, urethane, carbamate, isophorones and/or isocyanides, and one or more photoinitiators.

In addition, said varnish can comprise the varnish comprises at least one resin chosen from polyvinyl butyral resins, shellac resins, elemi resins, alkyd resins, polyester resins; tosylamide/epoxy resins; epoxy resins; acrylate resins; styrene-acrylate resins; polyurethane resins; sucrose benzoate; tosylamide/formaldehyde resins, toluenesulfonamide/formaldehyde resins, or polymers and/or copolymers of these various resins.

The addition of said aqueous chestnut extract appeared advantageous for tinted varnishes, containing at least one dye, pigments, pearlescent agents, glitter, metal particles or a mixture of these. It appeared that the stability of these varnishes is maintained over time. However, according to an alternative form of the invention, the varnish is a colorless varnish, containing neither pigment nor dye.

Advantageously, the cosmetic composition according to the invention can also comprise nail care products other than the chestnut extract chosen from vitamins A, B, C, E and their derivatives, calcium, silanes, alkyl sulfones, amino acids or peptides, in particular those containing sulfur, and their salts, hydrolyzed keratin, α-hydroxy acids, urea and its derivatives, oils of vegetable origin, extracts of plants other than the chestnut extract, and a combination of these.

The present invention also relates to the use of the cosmetic composition described above for improving the surface condition of the nail, in particular for nourishing and hydrating the nail. The use of this cosmetic care composition is a nontherapeutic use.

The advantage of a varnish, in comparison with a cream which needs to be applied daily, is that its action is longer lasting, it being possible for the varnish to remain on the nail for several days without being removed, in particular during washing of the hands.

### EXAMPLES

The following examples make it possible to illustrate the present invention in a nonlimiting way. In all the formulations, the concentration of the different constituents is expressed in % by weight of the total weight of the cosmetic composition.

### Tests Carried Out

Various cosmetic compositions in the form of a nail varnish of organic solvent-based varnish type or of aqueous varnish type were prepared and applied to nails. The tests below were carried out on these varnishes:

Gloss: 100 µm of varnish were applied to a sheet of Leneta type. After drying, the gloss was measured at an angle of incidence of 60° using a Minolta 268 glossmeter.

Flexibility: An aluminum sheet covered with a varnish of 300 µm wet is subjected to slow stamping and the depth of the deformation of the metal sheet is measured (ISO1520) (expressed in mm).

Hardness of the varnish: was measured using a Persoz pendulum on a glass sheet covered with a varnish with a thickness of 100 µm wet, after drying at ambient temperature, according to the standard ISO1522 (expressed in s).

Adhesion: a “cross hatch test” is carried out on a glass sheet. A grade of 0 corresponds to an absence of loss of adhesion. A grade of 5 corresponds to a total loss of adhesion.

Solids content: Between 0.5 and 1 g of varnish is poured into a dish. This dish is subsequently placed in an oven at 100° C. for 3 hours, then weighed in order to calculate the solids content of the varnish.

Stability: The varnishes are packaged in glass bottles and stored in an oven at 50° C. for 1 month. The color of the varnish is subsequently compared with a reference varnish that does not contain the active principle.

Viscosity: The viscosity of the product is measured at 25° C. using a Brookfield viscometer. The measurements are taken after rotating for one minute at 6 rev/min (Visco 1), then after one minute at 60 rev/min (Visco 2) and finally after one minute at 6 rev/min (Visco 3). The values are given in mPa.s.

Water content: The water contained in a formula is assayed by the Karl Fischer method using a Metrohm item of equipment of 915 KF Ti-Touch type and commercial assaying solutions.

Drying: After having applied a film of varnish with a thickness of 100 µm on a Leneta WDX card, the card is placed on a thermostatically controlled plate (35° C. ± 0.5° C.). The drying time is measured by virtue of a circular recorder placed on the film. This recorder has a moving rod describing a circle and leaving a plot on the film as long as the latter is not dry. The angular distance left by the plot is subsequently converted into time by virtue of a pre-established conversion table.

Yellowing stability: The products packaged in glass varnish bottles are left in an oven at 50° C. and at ambient temperature (20° C.) for a month for a comparative study of the stability. This stability is evaluated visually (color/yellowing). Shades not very loaded with pigment are particularly suitable for this type of monitoring.

Stability to syneresis: The products packaged in glass varnish bottles are left in an oven at 50° C. and at ambient temperature for a month for a comparative study of the stability. This stability is evaluated visually (release of solvent leaving a cylinder at the surface of the varnish). The purple shade (see below) is particularly suitable for this type of monitoring.

Stability to sedimentation: The products packaged in glass varnish bottles are left in an oven at 50° C. and at ambient temperature (20° C.) for a month for a comparative study of the stability. This stability is evaluated visually (presence of particles at the bottom of the bottle). The pearly shade is particularly suitable for this type of monitoring.

### Formulas Tested

The base compositions with rheological agent were prepared according to the formulations presented in [Table 1].

TABLE 1

| | Base without active principle | Base + 0.3% Recoverine® | Base + 0.5% Recoverine® |
|---|---|---|---|
| Butyl acetate | 42.57% | 42.27% | 42.07% |
| Ethyl acetate | 20.60% | 20.60% | 20.60% |
| Nitrocellulose | 14.00% | 14.00% | 14.00% |
| Polyester resin | 9.50% | 9.50% | 9.50% |
| Tributyl acetylcitrate | 6.00% | 6.00% | 6.00% |
| Isopropyl alcohol | 6.00% | 6.00% | 6.00% |
| Stearalkonium bentonite | 1.30% | 1.30% | 1.30% |
| Phosphoric acid | 0.03% | 0.03% | 0.03% |
| Recoverine® | 0.00% | 0.30% | 0.50% |

The polyester resin is an adipic acid/neopentyl glycol/trimellitic anhydride copolymer diluted to 70% in butyl acetate.

The nitrocellulose is 70% in isopropanol.

The aqueous chestnut extract Recoverine® comprises approximately 10% of active dry matter composed predominantly of rhamnogalacturonans and of uronic acids.

### Shades

The cosmetic compositions according to the invention were tested according to different shades, by means of dyes and/or pigments (alone or as a mixture with titanium dioxide) added to the base composition above, with or without chestnut extract:

Pearly shade: shade containing a coloring solution and mother-of-pearl at approximately 1.5%. Visual inspection of the mothers-of-pearl makes it possible to know if the product is more sensitive to sedimentation (in comparison with a reference which is in this instance the same varnish composition, without Recoverine®).

Purple shade: shade containing 2% of a mixture of pigments (titanium dioxide, Red 34, ferric ferrocyanide blue) making it possible to check the stability of the product, in particular with respect to the release of solvent.

Pink shade: shade containing a mixture of pigments (titanium dioxide, Red 6). This type of shade, with a low load of pigment (0.8%), is conventionally sensitive to syneresis and to yellowing.

Red shade: shade containing 2% of a mixture of pigments predominant in Red 6 and titanium dioxide in a small amount.

Beige shade: shade with very low load of pigment (0.15%) (titanium dioxide and red iron oxide), making possible a subdued application while contributing nail strengthening care. This shade makes it possible to observe a possible sensitivity to yellowing.

### Example 1: Stability After 1 Month

Different tinted compositions were prepared and observed after 1 month at ambient temperature (20° C.) and also at 50° C. The results are presented in [Table 2] below.

TABLE 2

| Storage conditions | | Ambient temperature (20° C.) | 50° C. |
|---|---|---|---|
| Pink shade | 0.0% Recoverine® | Nothing to report | Slight syneresis |
| | 0.3% Recoverine® | Nothing to report | Slight syneresis |
| | 0.5% Recoverine® | Nothing to report | Slight syneresis |
| Purple shade | 0.0% Recoverine® | Nothing to report | Absence of syneresis |
| | 0.3% Recoverine® | Nothing to report | Absence of syneresis |
| | 0.5% Recoverine® | Nothing to report | Slight syneresis |
| Pearly shade | 0.0% Recoverine® | Nothing to report | Absence of sedimentation |
| | 0.3% Recoverine® | Nothing to report | Absence of sedimentation |
| | 0.5% Recoverine® | Nothing to report | Absence of sedimentation |
| Beige shade | 0.0% | Nothing to report | No yellowing |
| | 0.5% | Nothing to report | No yellowing |

The beige shade was used to prove, via the test panel (see example 5 below), that the addition of active principle to the varnish provides a real benefit for the nail and to monitor a possible phenomenon of yellowing over time.

These results show us that the presence of the chestnut extract does not detrimentally affect the stability of the shades even if they are subjected to extreme conditions since the behaviors with 0.3% and 0.5% of chestnut extract are similar to the results obtained with the shades without active principles.

The purple shade, regarded as a generally critical shade for evaluating the stability of products with regard to syneresis, shows a slight syneresis at 50° C. when 0.5% of Recoverine® is added. This behavior shows that 0.5% appears to be the limiting concentration for use of this aqueous extract in solvent-based varnishes having thixotropic properties. This phenomenon is due to the presence of water in the active principle as supplied by SILAB: this is because this water promotes a higher thixotropic index by acting as activator of the thixotropic agent.

### Example 2: Rheology

After one month in an oven in darkness and at a temperature of 50° C., the results obtained on three different tinted compositions (pink, purple sensitive to syneresis, red and pearly red sensitive to sedimentation) prepared according to the base composition of example 1 (with 2% of pigment) are presented in [Table 3]:

TABLE 3

| Shade | Duration | Recoverine® content | V₁ (mPa.s) | V₂ (mPa.s) | V₃ (mPa.s) | Thixotropic index |
|---|---|---|---|---|---|---|
| PINK shade | T₀ | 0% | 2646 | 714 | 1446 | 2.02 |
| | | 0.3% | 2685 | 760 | 1480 | 1.95 |
| | | 0.5% | 3272 | 728 | 1680 | 2.33 |
| | T_{0 + 1} month | 0% | 1440 | 470 | 860 | 1.83 |
| | | 0.3% | 1440 | 486 | 880 | 1.81 |
| | | 0.5% | 1440 | 448 | 880 | 1.96 |
| PURPLE shade | T₀ | 0% | 2861 | 722 | 1780 | 2.46 |
| | | 0.3% | 3398 | 772 | 1960 | 2.54 |
| | | 0.5% | 3834 | 731 | 2400 | 3.28 |
| | | 0% | 1580 | 516 | 920 | 1.78 |
| | T_{0 + 1} month | 0.3% | 1720 | 524 | 940 | 1.79 |
| | | 0.5% | 1840 | 524 | 1220 | 2.33 |
| PEARLY shade | T₀ | 0% | 2600 | 690 | 1120 | 1.62 |
| | | 0.3% | 2517 | 705 | 1080 | 1.53 |
| | | 0.5% | 2518 | 687 | 1020 | 1.48 |
| | T_{0 + 1} month | 0% | 1460 | 470 | 680 | 1.45 |
| | | 0.3% | 1360 | 434 | 620 | 1.43 |
| | | 0.5% | 1340 | 410 | 700 | 1.71 |
| RED shade | T₀ | 0% | 3140 | 1206 | 1900 | 1.58 |
| | | 0.3% | 2600 | 1120 | 1820 | 1.62 |
| | | 0.5% | 2102 | 1034 | 1396 | 1.35 |

(V₁: 6 rev/min for 1 min; V₂: 60 rev/min for 1 min; V₃: 6 rev/min for 1 min - thixotropic index = V₃/V₂).

The values obtained are entirely acceptable: the chestnut extract, at the concentrations tested, does not adversely affect the stability of the varnish.

### Example 3: General Properties

All the properties tested by comparing a tinted composition according to example 1 (red shade - containing 2% by weight of pigment) without Recoverine® and with 0.3% of Recoverine® are presented in this example.

The study was also carried out on the base composition alone (without addition of pigment) with 0.3% of or without Recoverine®: see [Table 4].

TABLE 4

| | Base + 0% Recoverine® | | Base + 0.3% Recoverine® | |
|---|---|---|---|---|
| | Base alone | Red shade | Base alone | Red shade |
| Visco 1 (mPa.s) | 2420 | 3140 | 4100 | 2600 |
| Visco 2 (mPa.s) | 916 | 1206 | 1090 | 1120 |
| Visco 3 (mPa.s) | 1660 | 1900 | 2920 | 1820 |
| Thixo index | 1.81 | 1.58 | 2.68 | 1.63 |
| Gloss (%)⁽¹⁾ | 84.1 | 83.3 | 84.0 | 82.6 |
| Hardness(es)⁽¹⁾ | 198 | 199 | 191 | 208 |
| Adhesion | 0 | 0 | 0 | 0 |
| Solids content (%) | 31.58 | Nd | 31.32 | Nd |
| Water content (%) | 0.64 | Nd | 0.74 | Nd |
| Drying (min) | 2′30 | 3′30 | 3′15 | 3′00 |
| Stamping (mm) | 4.70 | 4.45 | 5.20 | 4.45 |
| Folding | OK | Minor cracks | OK | Minor cracks |

(1) mean value over 3 points - Nd: not determined

This comparative study shows us that the only notable difference occurs with the base composition alone, with or without the chestnut extract. This phenomenon of increase in the rheology (viscosity/thixotropy) is due to the presence of water in this aqueous extract, as indicated above. Water is known to be a very good activator of clays (used to regulate the rheology of nail varnishes) but its action is not long lasting. It may furthermore be noticed that the addition of pigments considerably reduces the action of water on the clays.

For all the other parameters measured, the results are satisfactory; the chestnut extract in these varnish compositions does not negatively affect the properties.

### Example 4: Aqueous Varnish

As the chestnut extract Recoverine® is an aqueous extract, it can be altogether incorporated in an aqueous-based varnish. The formulations can, for example, be in the following ranges of concentrations by weight:
H₂O 50.0% - 90.0%
Viscosifying film-forming carbomer 0.1% - 2.0%
Trisodium phosphate 2.0% - 4.0%
Glycerol 5.0% - 15.0%
Propylene glycol 1.0% - 10%
Phenoxyethanol 0.5% - 1.5%
Recoverine® 0.1 % - 10.0%

A test carried out with an aqueous varnish composition containing 3% of Recoverine® applied to the nails twice weekly for 15 days showed a notable improvement in the quality of the nails: the nails were more hydrated and less brittle.

### Example 5: Consumer Test

A usage test was carried out on a panel of 15 volunteers, for 2 weeks. The solvent-based varnish composition (in accordance with that of example 1 with 0.15% of pigments - beige shade) containing 0.5% of Recoverine® was applied twice weekly to the nails of all the fingers in two coats. Each panelist evaluated the properties at D0, at 7 days (D7) and at 14 days (D14). It was compared with varnish compositions containing either α-hydroxy acids or a mixture of α-hydroxy acids and of polyhydroxy acids which are known to improve the condition of keratin materials.

At D0 and D14, the panelists assigned a grade from 1 to 10 on the quality of their nails before and after treatment. The panelists noticed (p < 0.05) a notable improvement in the condition of the nails, more particularly:
Nails less fragile → 87% of people
Nails less brittle → 87% of people
Nails which split less → 87% of people
Nails less soft → 80% of people
Nails better hydrated → 80% of people
Nails less rough → 80% of people.

No one noticed any difficulty in applying the varnish composition, or any reduction in the gloss. Only the drying time on the nail was slightly longer.

Nevertheless, the tested composition surprisingly improves the hydration of the nails, from the 7th day of application.

1. A cosmetic nail care composition existing in the form of a varnish to be applied to the surface of the nail, comprising:
at least one rhamnogalacturonan; and
at least one uronic acid.

2. The composition as claimed in claim 1, wherein said composition contains a chestnut extract.

3. The composition as claimed in claim 2, wherein said composition comprises an amount of chestnut extract of between 0.01% and 10% by weight.

4. The composition as claimed in claim 2, wherein the varnish is a solvent-based varnish comprising an organic solvent medium, at least one film-forming agent, at least one plasticizer, at least one resin and optionally a rheology agent and contains said chestnut extract in a proportion of between 0.01% and 1% by weight.

5. The composition as claimed in claim 4, wherein the organic solvent medium comprises one or more solvents chosen from the group consisting of: short-chain esters having from 3 to 8 carbon atoms in total, such as ethyl acetate, methyl acetate, propyl acetate, isopropyl acetate, butyl acetate, isopentyl acetate, methoxypropyl acetate, butyl lactate, ketones which are liquid at ambient temperature, alcohols, such as ethanol, isopropanol, butanol or diacetone alcohol, linear or branched aliphatic hydrocarbons which are saturated, such as alkanes, or unsaturated, and liquid at ambient temperature, aromatic hydrocarbons, such as toluene or xylene, and their mixtures.

6. The composition as claimed in claim 5, wherein the organic solvent medium comprises ethyl acetate and/or butyl acetate and/or at least one alcohol chosen from ethanol, isopropanol and butanol.

7. The composition as claimed in claim 2, wherein the varnish is an aqueous-based varnish comprising an aqueous solvent medium, at least one film-forming agent, at least one plasticizer, at least one resin and optionally a rheology agent and contains said chestnut extract in a proportion of between 0.01% and 10% by weight.

8. The composition as claimed in claim 1, wherein the varnish comprises at least one cellulose film-forming agent chosen from nitrocellulose, carboxymethylcellulose, hydroxyethylcellulose, hydroxypropylcellulose, cellulose acetate butyrate and/or cellulose acetate propionate.

9. The composition as claimed in claim 1, wherein the varnish comprises one or more monomers or oligomers based on (meth)acrylate, urethane, carbamate, isophorones and/or isocyanides, and one or more photoinitiators.

10. The composition as claimed in claim 4, wherein the varnish comprises at least one resin chosen from polyvinyl butyral resins, shellac resins, elemi resins, alkyd resins, polyester resins; tosylamide/epoxy resins; epoxy resins; acrylate resins; styrene-acrylate resins; polyurethane resins; sucrose benzoate; tosylamide/formaldehyde resins, toluenesulfonamide/formaldehyde resins, or polymers and/or copolymers of these various resins.

11. The composition as claimed in claim 1, wherein the varnish is a tinted varnish, containing at least one dye, pigments, pearlescent agents, glitter, metal particles or a mixture of these.

12. The composition as claimed in claim 1, wherein said composition comprises one or more nail care products other than the chestnut extract chosen from vitamins A, B, C, E and their derivatives, calcium, silanes, alkyl sulfones, amino acids or peptides.

13. The composition as claimed in claim 1, said composition improves the surface condition of the nail.

14. The composition as claimed in claim 13, wherein said composition nourishes and hydrates the nail.

15. The composition as claimed in claim 2, wherein said composition contains a Castanea sativa extract, comprising rhamnogalacturonans and uronic acids.

16. The composition as claimed in claim 3, wherein said composition comprises an amount of chestnut extract of between 0.05% and 8% by weight.

17. The composition as claimed in claim 3, wherein said composition comprises an amount of chestnut extract of between 0.1% and 6% by weight.

18. The composition as claimed in claim 4, wherein the varnish is a solvent-based varnish comprising an organic solvent medium, at least one film-forming agent, at least one plasticizer, at least one resin and optionally a rheology agent and contains said chestnut extract in a proportion of between 0.05% and 0.8% by weight.

19. The composition as claimed in claim 4, wherein the varnish is a solvent-based varnish comprising an organic solvent medium, at least one film-forming agent, at least one plasticizer, at least one resin and optionally a rheology agent and contains said chestnut extract in a proportion of between 0.1% and 0.6% by weight.

20. The composition as claimed in claim 7, wherein the varnish is an aqueous-based varnish comprising an aqueous solvent medium, at least one film-forming agent, at least one plasticizer, at least one resin and optionally a rheology agent and contains said chestnut extract in a proportion of between 0.05% and 8% by weight.

21. The composition as claimed in claim 7, wherein the varnish is an aqueous-based varnish comprising an aqueous solvent medium, at least one film-forming agent, at least one plasticizer, at least one resin and optionally a rheology agent and contains said chestnut extract in a proportion of between 0.1% and 6% by weight.

22. The composition as claimed in claim 12, wherein said composition comprises one or more nail care products other than the chestnut extract chosen from vitamins A, B, C, E and their derivatives, calcium, silanes, alkyl sulfones, amino acids or peptides which include sulfur, and their salts, hydrolyzed keratin, α-hydroxy acids, urea and its derivatives, oils of vegetable origin, extracts of plants other than the chestnut extract, and a combination of these.
